(19) United States
(12) Patent Application Publication
(10) Pub. No.: US 2021/0228720 A1
(43) Pub. Date: Jul. 29, 2021

(54) ABSORBENT BIOPHOTONIC FIBER SYSTEM

(71) Applicant: KLOX TECHNOLOGIES INC., Laval (CA)

(72) Inventors: David OHAYON, Dollard-des-Ormeaux (CA); Carlo BELLINI, Mont-Royal (CA); Jason GUGLIUZZA, Yorkshire (GB); Nikolaos LOUPIS, Athens (GR); Remigio PIERGALLINI, Grottammare (IT); Abdellatif CHENITE, Kirkland (CA)

(21) Appl. No.: 15/734,599

(22) PCT Filed: Jun. 5, 2019
(86) PCT No.: PCT/CA2019/050784

§ 371 Date: Dec. 3, 2020

Related U.S. Application Data
(60) Provisional application No. 62/680,947, filed on Jun. 5, 2018.
(60) Provisional application No. 62/768,702, filed on Nov. 16, 2018.

(51) Int. Cl. A61K 41/00; A61F 13/00; A61L 15/24; A61L 15/26; A61L 15/42; A61N 5/06; C09K 11/02; C09K 11/06

(52) U.S. Cl. CPC A61K 41/0057; A61N 2005/0662; A61L 15/24; A61L 15/26; A61L 15/42; A61N 5/062; A61N 5/0616; C09K 11/02; C09K 11/06; A61F 2013/00748; A61F 2013/00919; A61F 2013/00927; A61L 2300/41; A61L 2300/442; A61N 2005/0645; A61N 2005/0651; A61N 2005/0661; A61F 13/00012

(57) ABSTRACT

The present technology generally relates to an absorbent biophotonic fiber system and to articles comprising the absorbent biophotonic fiber system as well as to the potential uses thereof, such as, for example, in wound treatment.

### CROSS-REFERENCE TO RELATED APPLICATIONS

This application claims the benefit of U.S. Provisional Application No. 62/680,947, filed Jun. 5, 2018, and of U.S. Provisional Application No. 62/768,702, filed on Nov. 16, 2018, the disclosure of both of which is incorporated herein by reference in its entirety.

### FIELD OF TECHNOLOGY

The present disclosure generally relates to an absorbent biophotonic fiber system and to articles comprising the same as well as to the use of such system and articles in, for example, biophotonic treatments.

### BACKGROUND INFORMATION

Biophotonic compositions are now being recognized as having a wide range of applications in the medical, cosmetic and dental fields for use in surgeries, therapies and examinations. For example, biophotonic compositions have been used to treat skin and various tissue disorders as well as to promote wound healing. For these applications, biophotonic therapies have typically been achieved using biophotonic formulations and/or biophotonic compositions comprising light-absorbing molecules capable of absorbing and/or emitting light. These biophotonic formulations and/or compositions have typically been prepared and used as liquids or semi-liquids (e.g., gels, pastes, creams and the like). Due to their liquid and/or semi-liquid texture, some of these biophotonic formulations and/or compositions require a support/surface onto which they can be applied. Some of these liquid and semi-liquid biophotonic formulations and/or compositions may also have a tendency to spread upon contact with fluid.

Some biophotonic fibers wherein the light-absorbing molecules are integrated into a fiber material have been proposed (e.g., WO 2016/065488). Such biophotonic fibers alleviate some of the drawbacks observed with the biophotonic formulations and compositions.

Despite the biophotonic fibers known to date, there remains a need in the art for biophotonic fiber systems that provide additional and/or complementary features allowing to expand the scope of biophotonic products that can be created and as well as to expand the scope of therapeutical applications in which these biophotonic products can be used.

### SUMMARY OF DISCLOSURE

According to various aspects, the present disclosure relates to an absorbent biophotonic fiber system comprising: at least one biophotonic fiber component; and at least one absorbent component; wherein the at least one biophotonic fiber component is photo-stimulated upon exposure to light to emit fluorescence. In some implementations, the absorbent biophotonic fiber system is suitable for topical application. In some implementations, the biophotonic fiber system comprises biophotonic fibers embedded in the system.

According to various aspects, the present disclosure relates to the use of the absorbent biophotonic fiber system as defined herein for healing, management or treatment of a wound.

According to various aspects, the present disclosure relates to the use of the absorbent biophotonic fiber system as defined herein in the manufacture of an article for healing, management or treatment of a wound.

According to various aspects, the present disclosure relates to the use of the absorbent biophotonic fiber system as defined herein, wherein the article is a wound dressing.

According to various aspects, the present disclosure relates to the use of the absorbent biophotonic fiber system as defined herein in combination with a light source for healing, management or treatment of a wound.

According to various aspects, the present disclosure relates to an article of manufacture for healing, management or treatment of a wound, the article of manufacture comprising: at least one biophotonic fiber component comprising biophotonic fibers, wherein interstices are present between the biophotonic fibers; and at least one hydrogel component comprising a hydrogel; at least a portion of the hydrogel is present in the interstices.

According to various aspects, the present disclosure relates to the use of the article of manufacture as defined herein for healing, management or treatment of a wound. According to various aspects, the present disclosure relates to the use of the article of manufacture as defined herein, wherein the article is a wound dressing. According to various aspects, the present disclosure relates to the use of the article of manufacture as defined herein in combination with a light source for healing or treatment of a wound.

According to various aspects, the present disclosure relates to a method for wound healing, wound management or wound treatment, the method comprising: a) applying the absorbent biophotonic fiber system as defined herein or the article of manufacture as defined herein onto a wound; and b) illuminating the absorbent biophotonic fiber system or the article of manufacture according with actinic light for a time sufficient to achieve photoactivation of the embedded biophotonic fiber component.

According to various aspects, the present disclosure relates to a kit for healing, management, and/or treatment of a wound, the kit comprising: the absorbent biophotonic fiber system as described herein or the article of manufacture as described herein; and instructions for use of the kit in the treatment of the wound.

A kit for treatment of a wound, the kit comprising: the absorbent biophotonic fiber system as described herein or the article of manufacture as described herein; and a light source.

Other aspects and features of the present technology will become apparent to those ordinarily skilled in the art upon review of the following description of specific embodiments in conjunction with the accompanying drawings.

### BRIEF DESCRIPTION OF FIGURES

All features of embodiments which are described in this disclosure are not mutually exclusive and can be combined with one another. For example, elements of one embodiment can be utilized in the other embodiments without further mention. A detailed description of specific embodiments is provided herein below with reference to the accompanying drawings in which:

FIG. 1 is a schematic representation of a cross-section of an absorbent biophotonic fiber system according to one embodiment of the present technology.

FIG. 2 is a schematic representation of a cross-section of an absorbent biophotonic fiber system according to another embodiment of the present technology.

FIGS. 3A-3F show graphs of cytokine modulation of IL-6 in Dermal Human Fibroblast (DHF) cells treated with the absorbent biophotonic fiber system according to one embodiment of the present technology. FIG. 3A: 3 hours after illumination; FIG. 3B: 6 hours after illumination; FIG. 3C: 18 hours after illumination; FIG. 3D: 24 hours after illumination; FIG. 3E: 48 hours after illumination; and FIG. 3F: 72 hours after illumination.

FIGS. 4A-4F show graphs of cytokine modulation of IL-6 in DHF cells treated with another absorbent biophotonic fiber system as used in FIGS. 3A-3F; FIG. 4A: 3 hours after illumination; FIG. 4B: 6 hours after illumination; FIG. 4C: 18 hours after illumination; FIG. 4D: 24 hours after illumination; FIG. 4E: 48 hours after illumination; and FIG. 4F: 72 hours after illumination.

### DETAILED DESCRIPTION

The present technology is explained in greater detail below. This description is not intended to be a detailed catalog of all the different ways in which the technology may be implemented, or all the features that may be added to the present technology. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which variations and additions do not depart from the present technology. Hence, the following description is intended to illustrate some particular embodiments of the technology, and not to exhaustively specify all permutations, combinations and variations thereof.

As used herein, the singular form “a,” “an” and “the” include plural referents unless the context clearly dictates otherwise. The recitation herein of numerical ranges by endpoints is intended to include all numbers subsumed within that range (e.g., a recitation of 1 to 5 includes 1, 1.5, 2, 2.75, 3, 3.80, 4, 4.32, and 5).

The term “about” is used herein explicitly or not, every quantity given herein is meant to refer to the actual given value, and it is also meant to refer to the approximation to such given value that would reasonably be inferred based on the ordinary skill in the art, including equivalents and approximations due to the experimental and/or measurement conditions for such given value. For example, the term “about” in the context of a given value or range refers to a value or range that is within 20%, preferably within 15%, more preferably within 10%, more preferably within 9%, more preferably within 8%, more preferably within 7%, more preferably within 6%, and more preferably within 5% of the given value or range.

The expression “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The term “biophotonic” as used herein refers to the generation, manipulation, detection and application of photons in a biologically relevant context. As used herein, the expression “biophotonic composition” refers to a light-absorbing-molecules containing composition as described herein that may be illuminated to produce photons for biologically relevant applications. As used herein, the expression “biophotonic regimen” or “biophotonic treatment” or “biophotonic therapy” refers to the use of a combination of a biophotonic composition as defined herein and emitted wavelengths from a light source given at an illumination period of that biophotonic composition to activate the light-absorbing molecules within the biophotonic composition.

Terms and expressions “light-absorbing molecule”, “light-capturing molecule”, “photoactivating agent”, “chromophore” and “photoactivator” are used herein interchangeably. A light-absorbing molecule means a molecule or a complex of molecules, which when contacted by light irradiation, is capable of absorbing the light. The light-absorbing molecules readily undergo photoexcitation and in some instances can then transfer its energy to other molecules or emit it as light when transforming from an unstable condition and transforming back to the ground state (i.e., fluorescence). The emitted fluorescence energy can be transformed to other components of the composition or to the treatment site. Different wavelengths may have different complementary therapeutic effects on tissue.

The expression “hydrogel” as used herein is not to be considered as limited to gels which contain water, but extend generally to all hydrophilic gels and gel compositions, including those containing organic polymer and/or non-polymeric components in addition to water. Different hydrogels may have different % of water (w/w) that affects the water absorption capacity of the hydrogels. For example, a hydrogel with a lower % of water content will have a higher degree of water absorbency. In addition, the pH of the hydrogel may be adjusted accordingly.

The term “actinic light” as used herein refers to light energy emitted from a specific light source (e.g., lamp, LED, or laser, or variations thereof) and capable of being absorbed by matter (e.g., the light-absorbing molecule defined above). In some embodiments, the actinic light is visible light.

As used herein, the term “treated”, “managed” in expressions such as: “treated tissue”, “managed tissue”, “managed skin”, “treated skin” and “managed area/portion of the skin”, “treated area/portion of the skin”, “managed soft tissue” and “treated soft tissue”, refers to a skin or soft tissue surface or layer(s) onto which a method according to the embodiments of the present technology has been performed.

In some aspects of these embodiments, the expression “biological tissue” refers to any organ and tissue of a living system or organism. Examples of biological tissue include, but are not limited to: brain, the cerebellum, the spinal cord, the nerves, blood, heart, blood vessels, skin, hair, fat, cartilage, ligaments, tendons, ovaries, fallopian tubes, uterus, vagina, mammary glands, testes, vas deferens, seminal vesicles, prostate, salivary glands, esophagus, stomach, liver, gallbladder, pancreas, intestines, rectum, anus, kidneys, ureters, bladder, urethra, the pharynx, larynx, bronchi, diaphragm, hypothalamus, pituitary gland, pineal body or pineal gland, thyroid, parathyroid, adrenals (e.g., adrenal glands), lymph nodes and vessels, skeletal muscles, smooth muscles, cardiac muscle, peripheral nervous system, ears, eyes, nose, gums, scalp, and the like.

As used herein, the term “fiber” relates to a string or a thread or a filament used as a component of composite materials. Fibers may be used in the manufacture of other materials such as for example, but not limited to, fabrics.

As used herein, the expression “woven” refers to a material (e.g., fabric) that is formed by weaving. As used herein, the expression “non-woven” refers to a material (e.g., fabric) that is made from staple fibers (short) and long fibers (continuous long), bonded together by chemical, mechanical, heat or solvent treatment. The expression “non-woven” may be used herein to denote a material which is neither woven nor knitted (e.g., a felt). As used herein, a “felt” is a textile that is produced by matting, condensing and pressing fibers together. As used herein, the term “carding” refers to a mechanical process that disentangles, cleans and intermixes fibres to produce a continuous web or sliver suitable for subsequent processing. This is achieved by passing the fibers between differentially moving surfaces covered with card clothing. It breaks up locks and unorganised clumps of fiber and then aligns the individual fibers to be parallel with each other. Optionally, the article produced from the carding process may thereafter be subject to a further mechanical process referred to as “calendaring” whereby the carded article is subjected to one or more needle press operations, each of which may be a different number smooth, hooked or barbed needles, wherein the needles are repetitively inserted into and withdrawn from the carded article, and through which process the randomly orientated fibers are further interlocked subsequent to the completion of the carding process. With respect to non-woven material, the degree of composition of the calendared scrim may impact the rigidity and formability of the article to provide form and shape. For example, a cast that may be used for placement and removal from a patient.

As used herein the term “wound” refers to an injury in which skin is torn, cut, or punctured (i.e., an open wound), or where blunt force trauma causes a contusion (i.e., closed wound), or sutured wound. Open wounds can be classified according to the object that caused the wound: Incisions or incised wounds (including excisional wounds) are caused by a clean, sharp-edged object such as a knife, razor, or glass splinter. Lacerations are irregular tear-like wounds caused by some blunt trauma. Lacerations and incisions may appear linear (regular) or stellate (irregular). The term laceration is commonly misused in reference to incisions. Abrasions (grazes) are superficial wounds in which the topmost layer of the skin (the epidermis) is scraped off. Abrasions are often caused by a sliding fall onto a rough surface. Avulsions are injuries in which a body structure is forcibly detached from its normal point of insertion. A type of amputation where the extremity is pulled off rather than cut off. Puncture wounds are caused by an object puncturing the skin, such as a splinter, nail or needle. Penetration wounds are caused by an object such as a knife entering and coming out from the skin. Gunshot wounds are caused by a bullet or similar projectile driving into or through the body. There may be two wounds, one at the site of entry and one at the site of exit, generally referred to as a “through-and-through”. Wounds suffered from blast injuries. Closed wounds include: Hematomas (or blood tumor) which are caused by damage to a blood vessel that in turn causes blood to collect under the skin. Hematomas that originate from internal blood vessel pathology are petechiae, purpura, and ecchymosis. The different classifications are based on size. Hematomas that originate from an external source of trauma are contusions, also commonly called bruises. Crush injury are caused by a great or extreme amount of force applied over a long period of time. According to level of contamination, a wound can be classified as: a clean wound which is made under sterile conditions where there are no organisms present and the skin is likely to heal without complications. Contaminated wounds are usually resulting from accidental injury; there are pathogenic organisms and foreign bodies in the wound. Infected wounds are the wound with pathogenic organisms present and multiplying, exhibiting clinical signs of infection (yellow appearance, soreness, redness, oozing pus). Colonized wound is a chronic situation, containing pathogenic organisms, difficult to heal (i.e., bedsore). Wounds that are said to be acute are typically categorized as two main types: traumatic wounds and surgical wounds. Wounds that are said to be chronic are wounds that do not heal in an orderly set of stages and in a predictable amount of time the way most wounds do; wounds that do not heal within three months are often considered chronic. Chronic wounds seem to be detained in one or more of the phases of wound healing. As used herein, wounds include: venous ulcers (including venous leg ulcers), arterial ulcers, pre-ulcerative lesions, superficial ulcers, diabetic foot ulcers, and the like.

Wound dressings can be used to cover wounds in an effort to assist in the wound healing process and facilitate the management of the wound and promoting wound healing. An ideal wound dressing will possess certain characteristics in order to help with the wound healing process. Examples of desired characteristics include, the ability to retain and absorb moisture, allowing good permeation of gas, particularly for the supply of oxygen from the ambient air to the covered wound area and for removal of excess carbon dioxide from the wound area to the ambient air, as well as for control of bacterial growth. Biophotonic compositions have also been proposed to assist wound dressing in the promotion of healing of wounds such as chronic wounds (see, in particular, WO 2015/000058, incorporated herein, in its entirety, by reference).

The ability of a wound dressing to retain and absorb moisture may be achieved by including water absorbent materials in the wound dressing. Water absorbent materials useful for such application include, but are not limited to, hydrogels. Hydrogels are water-insoluble polymers having the ability to swell in water or aqueous solution and to retain a significant portion of water or aqueous solution within its structure. Hydrogels can posses a degree of flexibility similar to natural tissue, due to their significant water content and hydrogels can have various applications. Attempts have been made to improve upon certain properties of hydrogels, for example, to increase strength, water content, transparency, and permeability or biocompatibility properties, often with mixed results.

In one embodiment, the present technology relates to an absorbent biophotonic fiber system. In some implementations of this embodiment, the absorbent biophotonic fiber system is an absorbent biophotonic fiber dressing. In some instances, the absorbent biophotonic fiber dressing is an absorbent biophotonic fiber wound dressing.

In some embodiments, the absorbent biophotonic fiber system of the present technology comprises a biophotonic fiber component and an absorbent component. In some implementations of these embodiments, the biophotonic fiber component comprises a plurality of biophotonic fibers. In some implementations of these embodiments, the absorbent component is a hydrogel component.

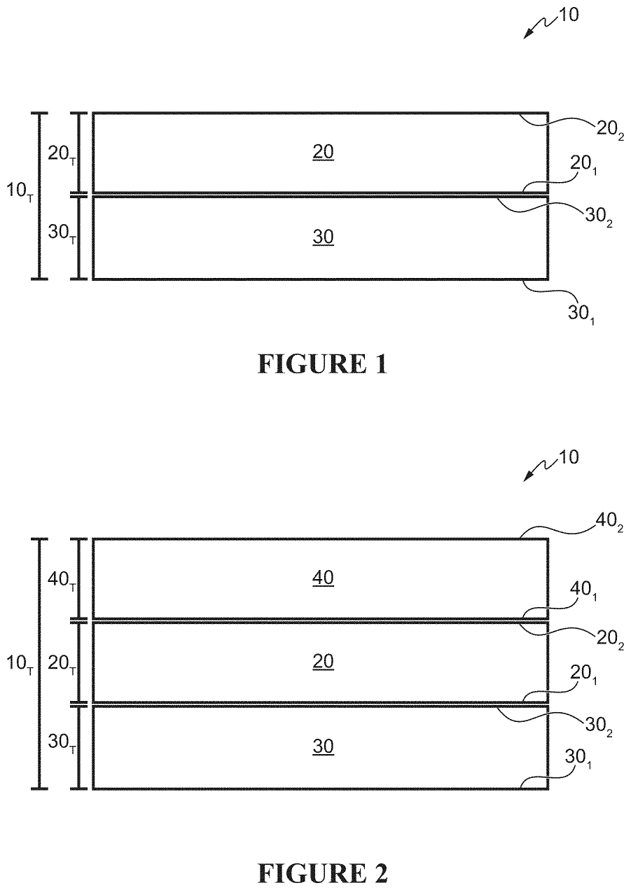

FIG. 1 illustrates an absorbent biophotonic fiber system according to one embodiment of the present technology. The absorbent biophotonic fiber system 10 has a thickness of 10_{T}. The absorbent biophotonic fiber system 10 comprises a biophotonic fiber component and an absorbent component 30. Both the biophotonic fiber component 20 and the absorbent component 30 have a tissue-facing surface 20₁, 30₁ and a non-tissue facing surface 20₂, 30₂. In some implementations, the tissue-facing surface 20₁ of the biophotonic fiber component 20 is in contact with the non-tissue facing surface 30₂ of the absorbent component 30. In situations where the absorbent biophotonic fiber system 10 is used as a wound dressing, preferably, the tissue-facing surface 30₂ of the absorbent component 30 is in direct contact with the tissue. The biophotonic fiber component 20 and the absorbent component 30 each have a thickness 20_{T}, 30_{T} spanning from their non-tissue facing surface 20₂, 30₂ to their tissue-facing surface 20₁, 30₁. In some instances, thickness 10_{T} of the absorbent biophotonic fiber system 10 ranges from between about 0.1 mm and about 10 mm, or between about 1 mm and about 9 mm, or between about 1 mm and about 6 mm, or between about 2 mm and about 6 mm. In some instances, thickness 20_{T} of the biophotonic fiber component 20 ranges from between about 0.1 mm and about 3 mm, or between 1 mm and about 3 mm, or between about 1 mm and about 2 mm, or less than about 2 mm, or less than about 1 mm. In some instances, thickness 30_{T} of the absorbent component 30 ranges from between about 0.1 mm and about 3 mm, or between 1 mm and about 3 mm, or between about 1 mm and about 2 mm, or less than about 2 mm, or less than about 1 mm. In some instances, thickness 10_{T} of the absorbent biophotonic fiber system 10 is the sum of the thickness of 20_{T} and 30_{T}.

In some embodiments, an adhesive (not shown) may be used to maintain the biophotonic fiber component 20 and the absorbent component 30 together. Examples of adhesive that may be used include, but are not limited to, acrylic adhesives which may be coated onto the either one (or both) of the tissue-facing surface 20₁ of the biophotonic fiber component 20 and the non-tissue facing surface 30₂ of the absorbent component 30. The adhesive may fully coat or only partially coat the surface onto which it is applied. When present only as a partial coating it is preferred that the adhesive forms a regular pattern. A partial coating may also be termed a discontinuous coating.

In some other embodiments, the hydrogel component itself is adhesive. In such embodiments, at least a portion of the biophotonic fiber component 20 is embedded in at least a portion of the absorbent component 30 as will be further described herein.

FIG. 2 illustrates an absorbent biophotonic fiber system 10 according to another embodiment of the present technology. In this embodiment, the absorbent biophotonic fiber system 10 comprises a biophotonic fiber component 20, a first absorbent component 30 and a second absorbent component 40. Each of the components 20, 30 and 40 comprises a tissue-facing surface 20₁, 30₁, 40₁ and a non-tissue facing surface 20₂, 30₂, 40₂. In this embodiment, the biophotonic fiber component 20 is disposed between (e.g., sandwiched) the first absorbent component 30 and the second absorbent component 40, wherein the tissue-facing surface 40₁ of the second absorbent component 40 is in contact with the non-tissue facing surface 20₂ of the biophotonic fiber component 20 and the tissue-facing surface 20₁ of the biophotonic fiber component 20 is in contact with the non-tissue facing surface 30₂ of the first absorbent component 30. The biophotonic fiber component 20, the first absorbent component 30 and the second absorbent component 40 each have a thickness 20_{T}, 30_{T}, 40_{T} spanning from their non-tissue facing surface 20₂, 30₂, 40₂ to their tissue-facing surface 20ₖ, 30ₖ, 40₁. In some instances, thickness 10_{T} of the absorbent biophotonic fiber system 10 ranges from between about 0.1 mm and about 10 mm, or between about 1 mm and about 9 mm, or between about 1 mm and about 6 mm, or between about 2 mm and about 6 mm. In some instances, thickness 20_{T} of the biophotonic fiber component 20 ranges from between about 0.1 mm and about 3 mm, or between 1 mm and about 3 mm, or between about 1 mm and about 2 mm, or less than about 2 mm, or less than about 1 mm. In some instances, thickness 30_{T} of the first absorbent component 30 ranges from between about 0.1 mm and about 3 mm, or between 1 mm and about 3 mm, or between about 1 mm and about 2 mm, or less than about 2 mm, or less than about 1 mm. In some instances, thickness 40_{T} of the second absorbent component 40 ranges from between about 0.1 mm and about 3 mm, or between 1 mm and about 3 mm, or between about 1 mm and about 2 mm, or less than about 2 mm, or less than about 1 mm. In some instances, the thickness 10_{T} of the absorbent biophotonic fiber system 10 is the sum of the thickness of 20_{T}, 30_{T} and ⁴0_{T}.

i) Biophotonic Fiber Component

In some embodiments, the biophotonic fiber component comprises biophotonic fibers. In some implementations of these embodiments, the biophotonic fibers form a biophotonic fabric or a biophotonic mesh. Biophotonic mesh may have a pre-determined wave pattern and spacing in the mesh to allow actinic light to pass into the mesh and the spacing may be adjusted accordingly. In yet another embodiment, the placement of fiber can be at random, or orderly fashion forming a scrim or other felt like orientation (similar to a cotton-candy texture). In some other implementations, the biophotonic fibers from a woven material. In some other implementations, the biophotonic fibers from a non-woven material.

The biophotonic fibers of the present disclosure comprise light-absorbing molecules that are photoactivatable or photostimulated by photoactivation or photostimulation of the biophotonic fibers. In some instances, the light-absorbing molecules are present on the surface of the biophotonic fibers (e.g., the biophotonic fibers are coated or sprayed with the light-absorbing molecules or the fibers are dipped into a composition or a formulation comprising the light-absorbing molecules). In other instances, the light-absorbing molecules are incorporated into the materials making the biophotonic fibers (e.g., the light-absorbing molecules are mixed/compounded with the materials making the biophotonic fibers). In some other implementations, the light-absorbing molecules are present both on the surface of the biophotonic fibers and incorporated/compounded into the materials making the biophotonic fibers.

In some instances, the biophotonic fibers are, but not limited to, synthetic fibers, natural fibers, and textile fibers. For example, synthetic fibers may be made from a polymer or a combination of different polymers. In some instances, the polymer is a thermoplastic polymer. In some implementations, the biophotonic fibers of the present disclosure are as described in WO2016/065488, incorporated herein in its entirety by reference.

In some instances, the polymer is acrylic, acrylonitrile butadiene styrene (ABS), polybenzimidazole (PBI), polycarbonate, polyether sulfone (PES), polyetherether ketone (PEEK), polyetherimide (PEI), polyethylene (PE), polyphenylene oxide (PPO), polyphenylene sulfide (PPS), polypropylene (PP), polystyrene, polyvinyl chloride (PVC), teflon, polybutylene, polyethylene terephthalate (PET), polybutylene terephthalate (PBT), nylon, polylactic acid (PLA), polymethyl methacrylate polyester, polyurethane, rayons, poly(methyl methacrylate) (PMMA), or from any mixture thereof.

In some other instances, the biophotonic fibers may be made from glycolic acid, copolymer lactide/glycolide, polyester polymer, copolymer polyglycolic acid/trimethylene carbonate, natural protein fiber, cellulose fiber, polyamide polymer, polymer of polypropylene, polymer of polyethylene, nylon, polymer of polylactic acid, polymer of polybutylene terephthalate, polyester, copolymer polyglycol, polybutylene, polymer of poly methyl methacrylate, or from any mixture thereof.

In some implementations, the biophotonic fibers of the present disclosure may be coextruded fibers that have two distinct polymers forming the biophotonic fibers, usually as a core-sheath or side-by-side.

In some implementations, the diameter of the biophotonic fibers (taken individually, monofilament) varies between about 15 microns and about 500 microns, between about 25 microns and about 500 microns, between about 50 microns and 400 microns, between about 50 microns and about 300 microns, preferably between about 50 microns and about 250 microns, preferably between about 75 microns and about 300 microns, and most preferably between about 75 microns and about 250 microns. In some specific implementations, the diameter of the biophotonic fibers defined herein is about 15 microns, about 20 microns, about 25 microns, about 50 microns, about 75 microns, about 100 microns, about 125 microns, about 150 microns, about 175 microns, about 200 microns, about 225 microns, about 250 microns, about 275 microns, about 300 microns, about 325 microns, about 350 microns, about 375 microns, about 400 microns, about 425 microns, about 450 microns, about 475 microns, about 500 microns. In some instances, the diameter of the biophotonic fibers defined herein (taken individually) is about 31 microns.

In some implementations, the biophotonic fibers have a linear mass density of between about 300 and about 480 Deniers, between about 410 and about 470 Deniers, between about 420 and about 460 Deniers, between about 420 and about 450 Deniers, or about 428 Deniers. As used herein, the term “Denier” refers to a unit of measure for the linear mass density of fibers, is defined as the mass in grams per 9000 meters.

In some embodiments, the biophotonic fibers of the present disclosure are prepared by an extrusion process wherein polymer pellets are melted and extruded and then pulled into a fiber while still hot. The fibers were dipped in Lurol Oil™/water solution (10%). The fibers are then spun onto a bobbin for storage and ease of use. In some instances, the biophotonic fibers of the present disclosure are prepared using a TEM co-rotating twin screw extruder.

In some implementations, the light-absorbing molecule is a chemical compound which, when exposed to the light is photoexcited and can then transfer its energy to other molecules or emit it as light, such as for example fluorescence. For example, in some instances, the light-absorbing molecule when photoexcited by the light may transfer its energy to enhance or accelerate light dispersion. Examples of light-absorbing molecules include, but are not limited to, fluorescent compounds (or stains) (also known as “fluorochromes” or “fluorophores” or “chromophores”). Other dye groups or dyes (biological and histological dyes, food colorings, carotenoids, and other dyes) can also be used. Suitable light-absorbing molecule can be those that are Generally Regarded As Safe (GRAS). In some instances, the light-absorbing molecule is a naturally-occurring chromophore, or is any small or large biological molecule capable of absorbing light and emitting one or more wavelength of light.

In certain implementations, the biophotonic fibers of the present disclosure comprise a first light-absorbing molecule. In some implementations, the first light-absorbing molecule absorbs at a wavelength in the range of the visible spectrum, such as at a wavelength of about 380 nm to about 1000 nm, about 380 nm to about 800 nm, about 380 nm to about 700 nm, about 400 nm to about 800 nm, or about 380 nm to about 600 nm. In other embodiments, the first light-absorbing molecule absorbs at a wavelength of about 200 nm to about 1000 nm, about 200 nm to about 800 nm, of about 200 nm to about 700 nm, of about 200 nm to about 600 nm or of about 200 nm to about 500 nm. In one embodiment, the first light-absorbing molecule absorbs at a wavelength of about 200 nm to about 600 nm. In some embodiments, the first light-absorbing molecule absorbs light at a wavelength of about 200 nm to about 300 nm, of about 250 nm to about 350 nm, of about 300 nm to about 400 nm, of about 350 nm to about 450 nm, of about 400 nm to about 500 nm, of about 450 nm to about 650 nm, of about 600 nm to about 700 nm, of about 650 nm to about 750 nm or of about 700 nm to about 800 nm. In some implementations, the light-absorbing molecule emits light within the range of about 400 nm and about 800 nm. In certain embodiments, the fluence delivered to the treatment areas may be between about 0.001 to about 60 J/cm², about 4 to about 60 J/cm², about 10 to about 60 J/cm², about 10 to about 50 J/cm², about 10 to about 40 J/cm², about 10 to about 30 J/cm², about 20 to about 40 J/cm², about 15 J/cm² to 25 J/cm², or about 10 to about 20 J/cm². In some embodiments, the fluence delivered to the treatment areas after 5 minutes of illumination is between about 33 J/cm² and about 45 J/cm², or between about 55 J/cm² and about 129 J/cm².

The biophotonic fibers disclosed herein may include at least one additional light-absorbing molecule. Combining light-absorbing molecules may increase photo-absorption by the combined light-absorbing molecules and enhance absorption and photo-biomodulation selectivity. Thus, in certain embodiments, the biophotonic fibers of the disclosure include more than one light-absorbing molecule.

In other implementations wherein the biophotonic fibers have the light-absorbing molecule on their surface (i.e., the surface of the fibers that is in contact with the surrounding environment of the fiber), such biophotonic fibers may be prepared by being sprayed with a light-absorbing molecule composition comprising one or more light-absorbing molecules and a carrier material.

In some specific examples, the light-absorbing molecule composition has a consistency that allows the fibers to be dipped into the composition. In some specific examples, the light-absorbing molecule composition is in a liquid or semi-liquid form. The carrier material may be any liquid or semi liquid material that is compatible with the light-absorbing molecule that is any material that does not affect the photoactive properties of the light-absorbing molecule, such as, for example, water. In some other specific examples, the light-absorbing molecule composition has a consistency that allows the light-absorbing molecule composition to be sprayed onto the fibers.

In the implementations wherein the biophotonic fibers have the light-absorbing molecule incorporated into the fibers, the biophotonic fibers are prepared by incorporating the light-absorbing molecule into the fiber composition. In some examples, the biophotonic fibers are prepared by extrusion. In some specific implementations, the biophotonic fibers are prepared by a process which uses spinning. The spinning may be wet, dry, dry jet-wet, melt, or gel. The polymer being spun may be converted into a fluid state. If the polymer is a thermoplastic then it may be melted, otherwise it may be dissolved in a solvent or may be chemically treated to form soluble or thermoplastic derivatives. The molten polymer is then forced through the spinneret, and then it cools to a rubbery state, and then a solidified state. If a polymer solution is used, then the solvent is removed after being forced through the spinneret. A composition of the light-absorbing molecule may be added to the polymer in the fluid state or to the melted polymer or to the polymer dissolved into a solvent. Melt spinning may be used for polymers that can be melted. The polymer having the light-absorbing molecules dispersed therein solidifies by cooling after being extruded from the spinneret.

The concentration of the light-absorbing molecule to be used may be selected based on the desired intensity and duration of the photoactivity to be emitted from the biophotonic fibers, and on the desired phototherapeutic, medical or cosmetic effect. For example, some dyes such as xanthene dyes reach a ‘saturation concentration’ after which further increases in concentration do not provide substantially higher emitted fluorescence. Further increasing the light-absorbing molecule concentration above the saturation concentration can reduce the amount of activating light passing through the biophotonic fibers. Therefore, if more fluorescence is required for a certain application than activating light, a high concentration of light-absorbing molecule can be used. However, if a balance is required between the emitted fluorescence and the activating light, a concentration close to or lower than the saturation concentration can be chosen.

Suitable light-absorbing molecule that may be used in the biophotonic fibers of the present disclosure include, but are not limited to the following: chlorophyll dyes, xanthene derivatives, methylene blue dyes and azo dyes. Examples of xanthene derivatives include, but are not limited to: eosin, eosin B (4′,5′-dibromo,2′,7′-dinitr-o-fluorescein, dianion); eosin Y; eosin Y (2′,4′,5′,7′-tetrabromo-fluorescein, dianion); eosin (2′,4′,5′,7′-tetrabromo-fluorescein, dianion); eosin (2′,4′,5′,7′-tetrabromo-fluorescein, dianion) methyl ester; eosin (2′,4′,5′,7′-tetrabromo-fluorescein, monoanion) p-isopropylbenzyl ester; eosin derivative (2′,7′-dibromo-fluorescein, dianion); eosin derivative (4′,5′-dibromo-fluorescein, dianion); eosin derivative (2′,7′-dichloro-fluorescein, dianion); eosin derivative (4′,5′-dichloro-fluorescein, dianion); eosin derivative (2′,7′-diiodo-fluorescein, dianion); eosin derivative (4′,5′-diiodo-fluorescein, dianion); eosin derivative (tribromo-fluorescein, dianion); eosin derivative (2′,4′,5′,7′-tetrachlor-o-fluorescein, dianion); eosin dicetylpyridinium chloride ion pair; erythrosin B (2′,4′,5′,7′-tetraiodo-fluorescein, dianion); erythrosin; erythrosin dianion; erythiosin B; fluorescein; fluorescein dianion; phloxin B (2′,4′,5′,7′-tetrabromo-3,4,5,6-tetrachloro-fluorescein, dianion); phloxin B (tetrachloro-tetrabromo-fluorescein); phloxine B; rose bengal (3,4,5,6-tetrachloro-2′,4′,5′,7′-tetraiodofluorescein, dianion); pyronin G, pyronin J, pyronin Y; Rhodamine dyes such as rhodamines that include, but are not limited to, 4,5-dibromo-rhodamine methyl ester; 4,5-dibromo-rhodamine n-butyl ester; rhodamine 101 methyl ester; rhodamine 123; rhodamine 6G; rhodamine 6G hexyl ester; tetrabromo-rhodamine 123; and tetramethyl-rhodamine ethyl ester.

In some embodiments, the light-absorbing molecule is an endogeneous molecules such as, but not limited to, vitamins. Examples of vitamins that may act as endogenous light-absorbing molecules include, vitamin B. In some instances, the endogenous light-absorbing molecule is vitamin B12. In some instances, the endogenous light-absorbing molecule is 7,8-Dimethyl-10-[(2S,3S,4R)-2,3,4,5-tetrahydroxypentyl]benzo[g]pteridine-2,4-dione. In some instances, the light-absorbing molecule is a naturally-occurring chromophore, or is any small or large biological molecule (e.g., peptides, polypeptides, nucleic acids, carbohydrates, or the like) capable of absorbing light and emitting one or more wavelength of light.

In certain embodiments, the biophotonic fibers of the present disclosure may include any of the light-absorbing molecules listed above, or a combination thereof, so as to provide a synergistic biophotonic effect. For example, the following synergistic combinations of light-absorbing molecules may be used: Eosin Y and Fluorescein; Fluorescein and Rose Bengal; Erythrosine in combination with Eosin Y, Rose Bengal or Fluorescein; Phloxine B in combination with one or more of Eosin Y, Rose Bengal, Fluorescein and Erythrosine; Eosin Y, Fluorescein and Rose Bengal.

In some examples, the light-absorbing molecule is present in the light-absorbing molecule composition at a concentration of about 100 g/L, about 50 g/L, about 10 g/L, about 5 g/L, about 1 g/L or about 0.1 g/L of the total volume. Preferably, the light-absorbing molecule is present in the light-absorbing molecule composition at a concentration of between about 10 g/L and about 100 g/L. In some instances, the light-absorbing molecule is present in the light-absorbing molecule composition at a concentration that is lower than 0.1 g/L, for example, the light-absorbing molecule is present in the light-absorbing molecule composition at a concentration in the milligram/L or in the microgram/L range.

In some embodiments, the biophotonic fibers of the present disclosure comprise a lubricant. In some instances, the lubricant is coated onto the biophotonic fibers of the present disclosure. In some instances, the lubricant is treatment oil, such as but not limited to Lurol Oil™.

In some implementations, the biophotonic fibers as defined herein may be woven into a fabric material resulting in a biophotonic fabric comprising a plurality of biophotonic fibers. In some implementations, the biophotonic fibers as defined herein may be bonded together by entangling the fibers mechanically, thermally or chemically to create a non-woven material. In some examples, the biophotonic woven or non-woven material may be used in the fabrication of an article of manufacture such as, but not limited to, a garment, an article of clothing, a wound dressing, a cast (e.g., surrounding a limb or the torso), a towel, bedding, and the like. In some implementation the garment may be a shirt, pants, glove, mask, socks, or the like. In some implementation, the non-woven fabric material may have a randomly or a non-randomly oriented fiber which influences the stiffness of the article. This may result in the article to have the ability to take on a particular shape such as for example when the article is a cast.

In some implementations, the biophotonic fibers as defined herein may be woven into a mesh resulting in a biophotonic mesh. As used herein, the expression “biophotonic mesh” refers to a loosely woven sheet of biophotonic fibers.

In the implementations wherein the light-absorbing molecules are compounded with the polymer of the fibers, the compounded polymer or the mesh made from such fibers is also photoactivatable. Whereas in the implementations wherein the light-absorbing molecules are not compounded with the polymer of the fibers, the fabric or the mesh made from such fibers may be coated or dipped or sprayed with a light-absorbing molecule composition to render the fabric photoactivatable.

In some other examples, the biophotonic fibers may be a non-woven biophotonic fabric or biophotonic mesh. Such biophotonic fabric and biophotonic mesh may be produced by depositing extruded, spun filaments onto a collecting belt in a uniform random manner followed by bonding the fibers. The fibers may be separated during the web laying process by air jets or electrostatic charges. The collecting surface is usually perforated to prevent the air stream from deflecting and carrying the fibers in an uncontrolled manner. Bonding imparts strength and integrity to the web by applying heated rolls or hot needles to partially melt the polymer and fuse the fibers together. In general, high molecular weight and broad molecular weight distribution polymers such as, but not limited to, polypropylene, polyester, polyethylene, polyethylene terephthalate, nylon, polyurethane, and rayons may be used in the manufacture of spunbound fabrics. In some instances, the biophotonic fabrics or biophotonic mesh may be composed of a mixture of polymers. A lower melting polymer can function as the binder which may be a separate fiber interspersed with higher melting fibers, or two polymers may be combined into a single fiber type. In the latter case the so-called bi-component fibers possess a lower melting component, which acts as a sheath covering over a higher melting core. Bicomponent fibers may also spun by extrusion of two adjacent polymers.

In some instances, spunbonding may combine fiber spinning with web formation by placing the bonding device in line with spinning In some arrangements the web may be bonded in a separate step. The spinning process may be similar to the production of continuous filament yarns and may utilize similar extruder conditions for a given polymer. Fibers are formed as the molten polymer exits the spinnerets and is quenched by cool air. The objective of the process is to produce a wide web and, therefore, many spinnerets are placed side by side to generate sufficient fibers across the total width.

Before deposition on a moving belt or screen, the output of a spinneret usually includes a plurality of individual filaments which must be attenuated to orient molecular chains within the fibers to increase fiber strength and decrease extensibility. This is accomplished by rapidly stretching the plastic fibers immediately after exiting the spinneret. In practice the fibers are accelerated either mechanically or pneumatically. The web is formed by the pneumatic deposition of the filament bundles onto the moving belt. A pneumatic gun uses high-pressure air to move the filaments through a constricted area of lower pressure, but higher velocity as in a venturi tube. In order for the web to achieve maximum uniformity and cover, individual filaments are separated before reaching the belt. This is accomplished by inducing an electrostatic charge onto the bundle while under tension and before deposition. The charge may be induced triboelectrically or by applying a high voltage charge.

The belt is usually made of an electrically grounded conductive wire. Upon deposition, the belt discharges the filaments. Webs produced by spinning linearly arranged filaments through a so-called slot die eliminating the need for such bundle separating devices.

Many methods can be used to bond the fibers in the spun web. These include mechanical needling, thermal bonding, and chemical bonding. The last two may bond large regions (area bonding) or small regions (point bonding) of the web by fusion or adhesion of fibers. Point bonding results in the fusion of fibers at points, with fibers between the point bonds remaining relatively free. Other methods used with staple fiber webs, but not routinely with continuous filament webs include stitch bonding, ultrasonic fusing, and hydraulic entanglement.

In some embodiments, the biophotonic fabrics and the biophotonic mesh of the present technology have interstices present between the biophotonic fibers making up the biophotonic fabrics or the biophotonic mesh.

In some embodiments, the biophotonic fibers of the present technology are bundled into one or more pattern to give bundled fibers. In some instances, the fibers may be bundled into one or more patterns (observed cross-sectionally) to give the bundled fibers that may be woven into a mesh or scrim as discussed herein.

ii) Absorbent Component

In some embodiments, the absorbent component is a hydrogel component. In such embodiments, the hydrogel component comprises a hydrogel. In some implementations of these embodiments, the hydrogel comprises a plurality of apertures that span through the hydrogel. In some implementations of these embodiments, the hydrogel comprises water dispersed in a hydrophilic polymer matrix. In some instances, the hydrophilic polymer matrix is a cross-linked hydrophilic polymer of a hydrophilic monomer. In some implementations of these embodiments, the hydrogel component further comprises additives such as, but not limited to, plasticisers (e.g., organic plasticisers), surfactants, polymers, pH regulators, electrolytes, chloride sources, and any mixture thereof. The water content of the hydrogel may be from about 0% by weight to about 95% by weight of the hydrogel, optionally from about 10% by weight to about 95% by weight of the hydrogel. The water content of the hydrogel may be at least about 40% by weight, optionally at least about 50% by weight. The water content of the hydrogel may be from about 10% by weight to about 40% by weight. The water content of the hydrogel may be from about 50% by weight to about 95% by weight. The hydrogel has the capacity to absorb water; for example the hydrogel may have a water-absorption capacity of at least about 30% by weight, optionally at least about 100% by weight, optionally at least about 200% by weight, optionally at least about 300% by weight; optionally between about 300% by weight and about 10000% by weight.

In the embodiments wherein the hydrogel comprises a plurality of apertures, the plurality of apertures enables the retaining of moisture into the hydrogel component. In situations where the absorbent biophotonic fiber system is used in a wound dressing, it may be desirable to maintain a moist wound environment for prolonged periods, over a wide range of wound exudation rates. When the exudation rate is high, the apertured layer of hydrogel swells and the size of the apertures decreases but not to the point that the apertures close. The layer of hydrogel is thereby able to remove wound fluid to prevent excessive moisture in the wound without removal of the hydrogel or blocking of the apertures in the hydrogel. Furthermore, the hydrogel may absorb moisture vapour and function as a humectant to preserve a moist wound contacting surface. The hydrogel may include one or more additional ingredients, which may be added to the pre-polymerisation mixture or the polymerised product, at the choice of the skilled worker. Such additional ingredients may be selected from additives, including, for example, water, organic plasticisers, surfactants, polymeric material (hydrophobic or hydrophilic in nature, including proteins, enzymes, naturally occurring polymers and gums), synthetic polymers with and without pendant carboxylic acids, electrolytes, pH regulators, colorants, chloride sources and mixtures thereof. The polymers can be natural polymers (e.g. xanthan gum), synthetic polymers (e.g., polyoxypropylene-polyoxyethylene block copolymer or poly-(methyl vinyl ether alt maleic anhydride)), or any combination thereof.

In the embodiments where the hydrogel comprises apertures, the apertures may be formed by first forming a sheet (or layer) of hydrogel and then cutting and removing a desired size and shape of holes from the sheet. In some instances, apertures are cut using a shaped die using techniques known in the art.

iii) Additional Materials

In some embodiments, the absorbent biophotonic fiber system of the present technology may comprise additional layers which may useful in, for example, the packaging, transport and/or storing of the system. For example, the absorbent biophotonic fiber system may comprise one or more liner on either or on both sides of the system. In some instances, such liners may be released liners which may be released from the absorbent biophotnic system prior to usage. Any commercially available release liners commonly used for such purposes can be utilized herein. Non-limiting examples of suitable release liners are polyethylene (PE) liners, polyester (PET) films, polyurethane (PU) films, or the like. In some implementations, a liner may be placed on one side of the absorbent biophotonic fiber system whereas the other side remains adhesive (i.e., sticky) or on the two sides of the absorbent biophotonic fiber system.

iv) Preparation and Method of Use

In some embodiments, the absorbent biophotonic fiber system of the present technology is prepared by disposing a layer of hydrogel precursor formulation on the biophotonic fibers or biophotonic fabric or biophotonic mesh of the present technology. In some instances portions of the applied hydrogel are cut and removed so as to form the apertures of the hydrogel. In some embodiments, the hydrogel is cut according to methods described in the art.

In the embodiments wherein the biophotonic fiber component is present between two hydrogel components (i.e., sandwiched between two hydrogel components), the apertures of the hydrogel have a thickness that allows light to reach the light-absorbing molecules embedded in the fibers of the biophotonic fibers and for the light emitted by the light-absorbing molecules to exit the hydrogel component.

In some embodiments, at least a portion of the biophotonic fiber component may be embedded in at least a portion of the hydrogel component such that hydrogel is present in the interstices formed between the biophotonic fibers.

In some embodiments, the interstices present between the biophotonic fibers are sufficiently large to facilitate evaporation of moisture from the hydrogel component. In some embodiments, the apertures of the hydrogel are sufficiently large to facilitate evaporation of moisture. It is thought that a high evaporation rate contributes to minimising the swelling of the hydrogel around the apertures and consequently decreasing the extent of aperture closure. In some instances, the apertures in the hydrogel facilitate traveling of the light emitted on the absorbent biophotonic fiber system as well as traveling of the light emitted by the biophotonic fibers out of the absorbent biophotonic fiber system. For example, and referring to FIG. 2, light (e.g., emitted by an actinic light source) reaching the non-tissue facing surface 40_{T} of hydrogel component 40 travels through the hydrogel of the hydrogel component 40 in part through the apertures of the hydrogel so as to reach biophotonic fiber component 20. Light reaching biophotonic component 20 photoactivates the light-absorbing molecules present in/on the biophotonic fibers. The photo-activated light-absorbing molecules then emit fluorescence which reaches, in turn, the tissue-facing surface 20₁ of the biophotonic fiber component 20 and the non-tissue facing surface 30₂ of the hydrogel component 30. The fluorescence then travels through the hydrogel in part through the apertures to reach the tissue-facing surface 30₁ of the hydrogel component 30 and out of the absorbent biophotonic fiber system 10. Fluorescence emitted by the absorbent biophotonic fiber system 10 may be used for photonic treatment of a tissue such as, for example, a wound.

In some embodiments, the absorbent biophotonic fiber system of the present disclosure may have therapeutic and/or cosmetic and/or medical benefits. In some implementations of these embodiments, the absorbent biophotonic fiber system may be used to promote the prevention and/or treatment of a tissue or an organ and/or to treat a tissue or an organ of a subject in need of phototherapy. In some instances, the absorbent biophotonic fiber system may be used to promote wound healing. In this case, the absorbent biophotonic fiber system may be applied at wound site as deemed necessary by the physician or other health care providers, or home patient caregivers, or patients alone. In certain embodiments, the absorbent biophotonic fiber system may be used following wound closure to optimize scar revision. In this case, the absorbent biophotonic fiber system may be applied at regular intervals such as once a week, or at an interval deemed appropriate by the physician or other health care providers, or patients alone. Wounds that may be treated by the absorbent biophotonic fiber system of the present disclosure include, for example, injuries to the skin and subcutaneous tissue initiated in different ways (e.g., surgical site infection, pressure ulcers from extended bed rest, pressure ulcers containing tunnels, colonized or infected wounds, wounds induced by trauma or surgery, burns, ulcers linked to diabetes or venous insufficiency) and with varying characteristics, arterial wounds, arterial ulcers, ischemic ulcers. In certain embodiments, the present disclosure provides absorbent biophotonic fiber system for treating and/or promoting the healing of, for example, burns, incisions, excisions, lesions, lacerations, abrasions, puncture or penetrating wounds, surgical wounds, contusions, hematomas, crushing injuries, amputations, post-surgical wounds, sores and ulcers.

In certain embodiments, the absorbent biophotonic fiber systems of the present disclosure are used in conjunction with systemic or topical antibiotic treatment (such as, for examples: tetracycline, erythromycin, minocycline, doxycycline). In some implementations, the article of manufacture being composed of the absorbent biophotonic fiber systems of the present disclosure may be able to control bacterial growth, for example when used in the treatment of a wound to minimize undesirable clinical outcomes associated with bacterial colonized wounds.

In some embodiments, the biophotonic fibers and fabrics of the present disclosure may be used in a method for effecting phototherapy on a subject, such as on a tissue (e.g., wounded tissue) of the subject. Such method comprises the step of applying an absorbent biophotonic fiber system as defined herein onto the subject or onto the tissue in need of phototherapy and the step of illuminating the absorbent biophotonic fiber system with light having a wavelength that overlaps partially, or in full, with an absorption spectrum of the light-absorbing molecule. In such embodiments, a tissue-facing surface of a hydrogel component of the absorbent biophotonic fiber system is placed on the tissue of a subject (e.g., skin). Light is then shed on the exposed non-tissue facing surface of the absorbent biophotonic fiber system (e.g., either on a non-tissue facing surface of a biophotonic fiber component or on a non-tissue facing surface of a hydrogel component).

In the methods of the present disclosure, any source of actinic light can be used. Any type of halogen, LED or plasma arc lamp, or laser may be suitable. The primary characteristic of suitable sources of actinic light will be that they emit light in a wavelength (or wavelengths) appropriate for activating the one or more light-absorbing molecule present in the absorbent biophotonic fiber systems. In one embodiment, an argon laser is used. In another embodiment, a potassium-titanyl phosphate (KTP) laser (e.g. a GreenLight™ laser) is used. In yet another embodiment, a LED lamp such as a photocuring device is the source of the actinic light. In yet another embodiment, the source of the actinic light is a source of light having a wavelength between about 200 nm to 800 nm. In another embodiment, the source of the actinic light is a source of visible light having a wavelength between about 400 nm and 600 nm. In another embodiment, the source of the actinic light is a source of visible light having a wavelength between about 400 nm and 700 nm. In yet another embodiment, the source of the actinic light is blue light. In yet another embodiment, the source of the actinic light is red light. In yet another embodiment, the source of the actinic light is green light. In yet another embodiment, the source of actinic light is a mixed light, for example: red and blue light or red and green light, or the like.

Furthermore, the source of actinic light should have a suitable power density. Suitable power densities for non-collimated light sources (LED, halogen or plasma lamps) are in the range from about 0.1 mW/cm² to about 200 mW/cm². Suitable power densities for laser light sources are in the range from about 0.5 mW/cm² to about 0.8 mW/cm².

In some implementations, the light has an energy at the subject's skin surface of between about 0.001 mW/cm² and about 500 mW/cm², or 0.1-300 mW/cm², or 0.1-200 mW/cm², wherein the energy applied depends at least on the condition being treated, the wavelength of the light, the distance of the tissue from the light source and the thickness of the absorbent biophotonic fiber systems. In certain embodiments, the light at the subject's tissue is between about 1-40 mW/cm², or between about 20-60 mW/cm², or between about 40-80 mW/cm², or between about 60-100 mW/cm², or between about 80-120 mW/cm², or between about 100-140 mW/cm², or between about 30-180 mW/cm², or between about 120-160 mW/cm², or between about 140-180 mW/cm², or between about 160-200 mW/cm², or between about 110-240 mW/cm², or between about 110-150 mW/cm², or between about 190-240 mW/cm².

The activation of the light-absorbing molecules may take place almost immediately on illumination (femto- or pico seconds). A prolonged exposure period may be beneficial to exploit the synergistic effects of the absorbed, reflected and reemitted light of the biophotonic fibers and fabrics of the present disclosure and its interaction with the tissue being treated. In one embodiment, the time of exposure of absorbent biophotonic fiber systems to actinic light is a period between 0.01 minutes and 90 minutes. In another embodiment, the time of exposure of the absorbent biophotonic fiber systems to actinic light is a period between 1 minute and 5 minutes. In some other embodiments, the absorbent biophotonic fiber systems are illuminated for a period between 1 minute and 3 minutes. In certain embodiments, light is applied for a period of about 1-30 seconds, about 15-45 seconds, about 30-60 seconds, about 0.75-1.5 minutes, about 1-2 minutes, about 1.5-2.5 minutes, about 2-3 minutes, about 2.5-3.5 minutes, about 3-4 minutes, about 3.5-4.5 minutes, about 4-5 minutes, about 5-10 minutes, about 10-15 minutes, about 15-20 minutes, or about 20-30 minutes.

The treatment time may range up to about 90 minutes, about 80 minutes, about 70 minutes, about 60 minutes, about 50 minutes, about 40 minutes or about 30 minutes. It will be appreciated that the treatment time can be adjusted in order to maintain a dosage by adjusting the rate of fluence delivered to a treatment area. For example, the delivered fluence may be about 4 to about 60 J/cm², 4 to about 90 J/cm², 10 to about 90 J/cm², about 10 to about 60 J/cm², about 10 to about 50 J/cm², about 10 to about 40 J/cm², about 10 to about 30 J/cm², about 20 to about 40 J/cm², about 15 J/cm² to 25 J/cm², or about 10 to about 20 J/cm², or about 0.001 J/cm² to about 1 J/cm².

In certain embodiments, the absorbent biophotonic fiber systems may be re-illuminated at certain intervals. In yet another embodiment, the source of actinic light is in continuous motion over the treated area for the appropriate time of exposure. In yet another embodiment, the absorbent biophotonic fiber systems may be illuminated until the absorbent biophotonic fiber systems is at least partially photobleached or fully photobleached.

In certain embodiments, the light-absorbing molecules in the absorbent biophotonic fiber systems can be photoexcited by ambient light including from the sun and overhead lighting. In certain embodiments, the light-absorbing molecules can be photoactivated by light in the visible range of the electromagnetic spectrum. The light can be emitted by any light source such as sunlight, light bulb, an LED device, electronic display screens such as on a television, computer, telephone, mobile device, flashlights on mobile devices. In the methods of the present disclosure, any source of light can be used. For example, a combination of ambient light and direct sunlight or direct artificial light may be used. Ambient light can include overhead lighting such as LED bulbs, fluorescent bulbs, and indirect sunlight.

In the methods of the present disclosure, the absorbent biophotonic fiber systems may be removed from the tissue following application of light. In other embodiments, the absorbent biophotonic fiber systems may be left on the tissue for an extended period of time and re-activated or not with direct or ambient light at appropriate times to treat the condition.

In certain instances, the absorbent biophotonic fiber system of the present disclosure may be used in the manufacture of articles such as; medical devices (e.g., wound dressing or the like).

Identification of equivalent compositions, methods and kits are well within the skill of the ordinary practitioner and would require no more than routine experimentation, in light of the teachings of the present disclosure. Practice of the disclosure will be still more fully understood from the following examples, which are presented herein for illustration only and should not be construed as limiting the disclosure in any way.

### EXAMPLES

The examples below are given so as to illustrate the practice of various embodiments of the present technology. They are not intended to limit or define the entire scope of this technology. It should be appreciated that the technology is not limited to the particular embodiments described and illustrated herein but includes all modifications and variations falling within the scope of the disclosure as defined in the appended embodiments.

### Example 1

### FLuorescence Emission Properties of a Biophotonic Fiber Mesh

Light-absorbing molecules were incorporated into fibers made of polymer materials (i.e., polymer materials compounded with light-absorbing molecules). The compounding involved taking a polymer melt and adding the light-absorbing molecules in their solid form directly to the polymer melt, and then allowing the melt to cool. This process allowed the light-absorbing molecules to be integrated into the polymer fibers. The light-absorbing molecule to polymer ratio was selected so as to be dependent on the light-absorbing molecules used, for example: for Eosin Y, a 1% w/w ratio (in water) was used was used for the master chromophore batch. Biophotonic fibers were made of polypropylene polymer (PP), of polyethylene polymer (PE), nylon-6, or of polylactic acid polymer (PA), or of a combination thereof. Eosin Y or fluorescein or a combination of Eosin Y and fluorescein were used as light-absorbing molecules. The biophotonic fibers made of polyethylene were made into a 50/50 polyethylene core/polypropylene sheath. Biophotonic meshes were prepared by knitting biophotonic fibers so as to make 1 mm thick mesh with a width of 11 cm (1 mm mesh) and a 2 mm thick mesh with a width of 22 cm (2 mm mesh). The biophotonic meshes were assessed for their ability to emit fluorescence following illumination for 5 minutes at 5 cm using a KT-L™ Lamp. The results are presented in Tables 1 for the 1 mm thick mesh and in Table 2 for the 2 mm thick mesh.

TABLE 1
Fluorescence emission of a light-stimulated
biophotonic woven mesh (1 mm)

| | mW/cm² at 5 cm | | | |
|---|---|---|---|---|
| | 0 min | 5 min | J/cm² | % |
| Lamp (400-518 nm) | 20.55 | 21.27 | 6.35 | 72.9 |
| Fluoresc. (519-760 nm) | 8.44 | 6.53 | 2.33 | 26.7 |
| TOTAL (400-760 nm) | 28.99 | 27.80 | 8.68 | 99.7 |
| % Fluorescence | 29.1 | 23.5 | 27 | 26.8 |
| Purple (400-450 nm) | 9.70 | 9.37 | 2.90 | 33.3 |
| Blue (450-500 nm) | 10.83 | 11.87 | 3.44 | 39.5 |
| Green (500-570 nm) | 0.53 | 0.45 | 0.16 | 1.8 |
| Yellow (570-591 nm) | 2.32 | 1.57 | 0.59 | 6.7 |
| Orange (591-610 nm) | 2.42 | 1.76 | 0.64 | 7.4 |
| Red (610-700 nm) | 3.26 | 2.85 | 0.98 | 11.3 |
| TOTAL | 29.10 | 27.88 | 8.70 | 100.0% |

TABLE 2
Fluorescence emission of a light-stimulated
biophotonic woven mesh (2 mm)

| | mW/cm² at 5 cm | | | |
|---|---|---|---|---|
| | 0 min | 5 min | J/cm² | % |
| Lamp (400-518 nm) | 63.90 | 64.01 | 19.28 | 95.9 |
| Fluoresc. (519-760 nm) | 3.32 | 2.18 | 0.82 | 4.1 |
| TOTAL (400-760 nm) | 67.21 | 66.19 | 20.09 | 99.9 |
| % Fluorescence | 4.9 | 3.3 | 4.1 | 4.1 |
| Purple (400-450 nm) | 26.72 | 25.12 | 7.77 | 38.6 |
| Blue (450-500 nm) | 37.17 | 38.88 | 11.51 | 57.3 |
| Green (500-570 nm) | 0.47 | 0.28 | 0.10 | 0.5 |
| Yellow (570-591 nm) | 1.17 | 0.78 | 0.29 | 1.5 |
| Orange (591-610 nm) | 1.03 | 0.68 | 0.26 | 1.3 |
| Red (610-700 nm) | 0.67 | 0.46 | 0.17 | 0.8 |
| TOTAL | 67.25 | 66.22 | 20.11 | 100.0% |

Table 3 presents the fluorescence emitted by a biophotonic gel composition comprising 1% w/w Eosin Y that was illuminated for 5 minutes at 5 cm using a KT-L™ Lamp. From the data presented in Tables 1, 2 and 3 it can be observed that a photoactivated absorbent biophotonic fiber system emits fluorescence extending to a greater degree into the yellow, orange and red wavelengths compared to the fluorescence emitted by a biophotonic composition (gel).

TABLE 3
Fluorescence emission of a biophotonic gel comprising Eosin Y

| | mW/cm² at 5 cm | | | |
|---|---|---|---|---|
| | 0 min | 5 min | J/cm² | % |
| Lamp (400-518 nm) | 64.10 | 108.76 | 30.11 | 99.5 |
| Fluoresc. (519-760 nm) | 1.79 | 0.08 | 0.14 | 0.46 |
| TOTAL (400-760 nm) | 65.89 | 108.84 | 30.25 | 99.96 |
| % Fluorescence | 2.7 | 0.07 | 0.46 | 0.46 |
| Purple (400-450 nm) | 35.7 | 47.50 | 13.73 | 45.39 |
| Blue (450-500 nm) | 28.38 | 60.94 | 16.32 | 53.95 |
| Green (500-570 nm) | 0.93 | 0.38 | 0.16 | 0.53 |
| Yellow (570-591 nm) | 0.57 | 0.00 | 0.03 | 0.10 |
| Orange (591-610 nm) | 0.24 | 0.00 | 0.01 | 0.03 |
| Red (610-700 nm) | 0.04 | 0.00 | 0.00 | 0.00 |
| TOTAL | 65.90 | 108.84 | 30.25 | 100% |

### Example 2

### Fluorescence Emission Properties of a Biophotonic Carded Material

A non-woven biophotonic fiber material was created in order to determine if carding, laminating and/or calendaring of biophotonic fibers could fluoresce in the range between 2.2-2.6 J/cm². Nylon biophotonic fibers were made as outlined in Example 1. Multiple spools of equal weight of 300 deniers were produced. These were then cut to 2.5″ in length and 14 crimps. The fiber bundle was gripped between two rolls and forced into a small chamber or stuffing box. Carded materials of various weights (gsm) were produced having the following composition: Fiber to Rayon Fiber ratio: 50/50 (Nylon Eosin/Rayon) and 70/30 (Nylon Eosin/Rayon). The fiber bundles were then laminated and carded. These non-woven biophotonic fiber materials were assessed for their ability to emit fluorescence following illumination for 5 minutes at 5 cm using a KT-L™ Lamp set at 33 J/cm². The results are presented in Table 4.

TABLE 4
Fluorescence emitted by non-woven biophotonic fiber system

| Sam- | Fluorescence | | | | | | |
|---|---|---|---|---|---|---|---|
| ples | (J/cm²) | Purple | Blue | Green | Yellow | Orange | Red |
| 1* | 2.61 | 2.33 | 1.87 | 67.8 | 0.7 | 0.38 | 0.48 |
| 2* | 2.47 | 3.59 | 3.52 | 0.53 | 0.63 | 0.33 | 0.41 |
| 3* | 2.27 | 2.35 | 2.34 | 0.38 | 0.57 | 0.32 | 0.42 |
| 4* | 2.19 | 2.24 | 2.36 | 0.36 | 0.55 | 0.3 | 0.41 |
| 5** | 2.43 | 4.72 | 4.78 | 0.62 | 0.59 | 0.3 | 0.36 |
| 6** | 2.41 | 2.48 | 2.53 | 0.4 | 0.62 | 0.35 | 0.47 |
| 7** | 2.39 | 4.06 | 4.06 | 0.54 | 0.58 | 0.31 | 0.40 |
| 8** | 2.4 | 3.26 | 3.62 | 0.48 | 0.59 | 0.32 | 0.43 |

*Low basis weight: 100-129 gsm
**High basis weight: 135-150 gsm

### Example 3

### Fluorescence Emission Properties of a Hydrogel

A 67% hydrogel was prepared. The hydrogel was assessed for its ability to emit fluorescence following illumination for 5 mins at 5 cm using a KT-L™ Lamp. The results are presented in Table 5.

TABLE 5
Fluorescence emission of a light-stimulated
hydrogel (hydrogel only)

| | mW/cm² at 5 cm | | | |
|---|---|---|---|---|
| | 0 min | 5 min | J/cm² | % |
| Lamp (400-518 nm) | 122.71 | 121.33 | 36.61 | 99.9 |
| Fluoresc. (519-760 nm) | 0.09 | 0.08 | 0.02 | 0.1 |
| TOTAL (400-760 nm) | 122.79 | 36.63 | 36.63 | 100.0 |
| % Fluorescence | 0.07 | 0.22 | 0.05 | 0.1 |
| Purple (400-450 nm) | 50.19 | 14.63 | 14.63 | 39.9 |
| Blue (450-500 nm) | 72.15 | 21.88 | 21.88 | 59.7 |
| Green (500-570 nm) | 0.41 | 0.12 | 0.12 | 0.3 |
| Yellow (570-591 nm) | 0.00 | 0.00 | 0.00 | 0.0 |
| Orange (591-610 nm) | 0.00 | 0.00 | 0.00 | 0.0 |
| Red (610-700 nm) | 0.03 | 0.00 | 0.00 | 0.0 |
| TOTAL | 122.80 | 36.63 | 36.63 | 100.0% |

### Example 4

### Fluorescence Emission Properties of a Biophotonic Fiber Mesh with Hydrogel

The biophotonic fiber mesh with hydrogel was prepared by combining the hydrogel as described in Example 3 on top of a 1 mm biophotonic mesh as described in Example 1 and illuminating the hydrogel (i.e., hydrogel on top of mesh). The biophotonic fiber system was assessed for its ability to emit fluorescence following illumination for 5 minutes at 5 cm using a KT-L™ Lamp. The results are presented in Table 6.

TABLE 6
Fluorescence emission of a light-stimulated
biophotonic fiber system with hydrogel

| | mW/cm² at 5 cm | | | |
|---|---|---|---|---|
| | 0 min | 5 min | J/cm² | % |
| Lamp (400-518 nm) | 18.19 | 18.45 | 5.46 | 76.5 |
| Fluoresc. (519-760 nm) | 6.26 | 5.04 | 1.66 | 23.3 |
| TOTAL (400-760 nm) | 24.44 | 23.48 | 7.12 | 99.7 |
| % Fluorescence | 25.6 | 21.5 | 23.3 | 23.4 |
| Purple (400-450 nm) | 7.79 | 7.30 | 2.24 | 31.4 |
| Blue (450-500 nm) | 10.36 | 11.10 | 3.21 | 44.9 |
| Green (500-570 nm) | 0.59 | 0.51 | 0.16 | 2.2 |
| Yellow (570-591 nm) | 1.69 | 1.26 | 0.43 | 6.0 |
| Orange (591-610 nm) | 1.60 | 1.25 | 0.42 | 5.9 |
| Red (610-700 nm) | 2.46 | 2.09 | 0.69 | 9.6 |
| TOTAL | 24.52 | 23.54 | 7.14 | 100.0% |

A further biophotonic fiber system was prepared by placing a 1 mm biophotonic mesh as described in Example 1 on top of a hydrogel as described in Example 3 and by illuminating the biophotonic mesh (i.e., mesh on top of hydrogel). The biophotonic fiber system was assessed for its ability to emit fluorescence following illumination for 5 minutes at 5 cm using a KT-L™ Lamp. The results are presented in Table 7.

TABLE 7
Fluorescence emission of a light-stimulated
biophotonic fiber system with hydrogel

| | mW/cm² at 5 cm | | | |
|---|---|---|---|---|
| | 0 min | 5 min | J/cm² | % |
| Lamp (400-518 nm) | 15.33 | 15.82 | 5.23 | 79.1 |
| Fluoresc. (519-760 nm) | 2.86 | 2.25 | 1.38 | 20.8 |
| TOTAL (400-760 nm) | 18.18 | 18.06 | 6.61 | 99.8 |
| % Fluorescence | 15.7 | 12.4 | 21 | 20.8 |
| Purple (400-450 nm) | 6.68 | 6.44 | 2.23 | 33.7 |
| Blue (450-500 nm) | 8.64 | 9.37 | 3.00 | 45.2 |
| Green (500-570 nm) | 0.23 | 0.16 | 0.12 | 1.8 |
| Yellow (570-591 nm) | 0.85 | 0.63 | 0.35 | 5.3 |
| Orange (591-610 nm) | 0.95 | 0.73 | 0.36 | 5.4 |
| Red (610-700 nm) | 0.85 | 0.74 | 0.57 | 8.7 |
| TOTAL | 18.23 | 18.10 | 6.63 | 100.0% |

Example 5

### Fluorescence Emission Properties of a Biophotonic Carded Material with Hydrogel

The biophotonic carded material was prepared by combining the hydrogel as described in Example 3 with the absorbent biophotonic carded material as described in Example 2 and illuminating the hydrogel. The biophotonic carded material was assessed for its ability to emit fluorescence following illumination for 5 minutes at 5 cm using a KT-L™ Lamp set at 33 J/cm². The results are presented in Table 8.

TABLE 8
Fluorescence emission of a light-stimulated
biophotonic carded material with hydrogel

| Sam- | Fluorescence | | | | | | |
|---|---|---|---|---|---|---|---|
| ples | (J/cm²) | Purple | Blue | Green | Yellow | Orange | Red |
| 1* | 0.60 | 6.69 | 8.36 | 0.26 | 0.17 | 0.11 | 0.1 |
| 2* | 0.70 | 5.78 | 7.43 | 0.25 | 0.21 | 0.14 | 0.13 |
| 3* | 0.74 | 6.1 | 8.12 | 0.27 | 0.21 | 0.14 | 0.16 |
| 4* | 0.87 | 5.46 | 7.5 | 0.33 | 0.23 | 0.16 | 0.19 |
| 5** | 0.86 | 6.32 | 7.74 | 0.31 | 0.24 | 0.16 | 0.18 |
| 6** | 0.83 | 5.92 | 7.7 | 0.3 | 0.23 | 0.16 | 0.18 |
| 7** | 0.93 | 5.2 | 6.96 | 0.3 | 0.26 | 0.18 | 0.21 |
| 8 | 0.74 | 6.74 | 8.53 | 0.28 | 0.2 | 0.14 | 0.15 |

*Low basis weight: 100-129 gsm
**High basis weight: 135-150 gsm

### Example 6

### Anti-Inflammation Properties of an Absorbent Biophotonic Fiber System

An experiment was carried out in order to evaluate the anti-inflammatory effects of an absorbent biophotonic fiber system as defined in Example 1. Dermal human fibroblasts (DHF cells) were purchased from the American Type Culture Collection (ATCC, Manassas, Va., USA) and grown in fibroblast basal medium supplemented with fibroblast growth kit-low-serum (ATCC). Cells were passed at 80% confluence and used at third and fourth passages. Human recombinant IL-1α, β and the Elisa Kit were purchased from R&D systems (Minneapolis, Minn., USA). The XTT was purchased from Fisher Thermoscientific (Waltham, Mass., USA). The hydrogel dressing used was 30% H₂O with a 50/50 mesh. Prior to stimulation, DHF cells were seeded in 2, 2-well slides Lab-Tek (Thermo Scientific; Waltham, Mass., USA) at a density of 60,000 cells/well for assessment of pro-inflammatory cytokine IL-6 release. After 5-6 hours of incubation at 37° C. in a humidified 5% CO₂ environment, cells were pre-stimulated with a IL-1α/β cocktail at 20 ng/ml for 18 hours. The next day, the medium was replaced with PBS during the illumination's period. Fresh IL-1α/β medium was then added and a time course (3, 6, 18, 24, 48, 72 hours) was performed on IL-6 production. Supernatants were then collected at each time point and was analysed by Elisa kit. Illumination conditions: 5 min or 2 illuminations of 5 min with a pause of 1 min (5-1-5) at 5 cm distance from KT-L™ LED Lamp LBL-0129.1) Control untouched (cells were not exposed to either stimulation or treatment); 2) Control stimulated (cells were stimulated but not treated); 3) Biophotonic mesh +hydrogel dressing 50/50 (stimulated cells were treated with the dressing in direct contact); 4) Blank hydrogel without mesh (stimulated cells were treated with the blank in direct contact); and 5) Dexamethasone 5 μM (positive control as anti-inflammatory properties.). Another equal set of conditions was tested without exposing the cells to the light.

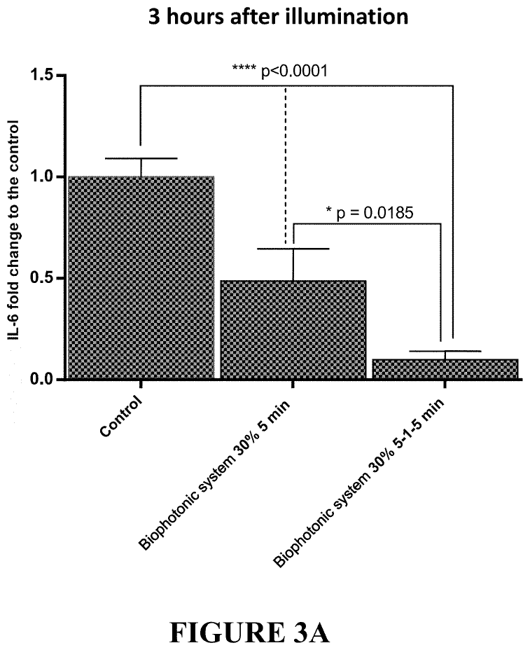

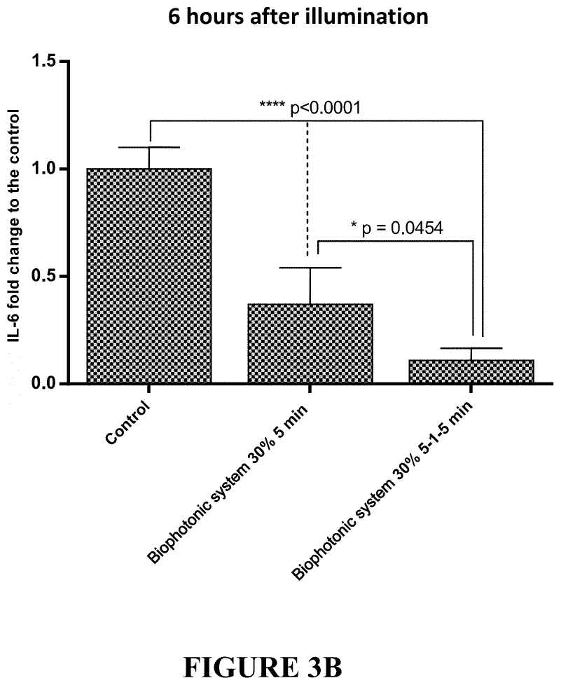

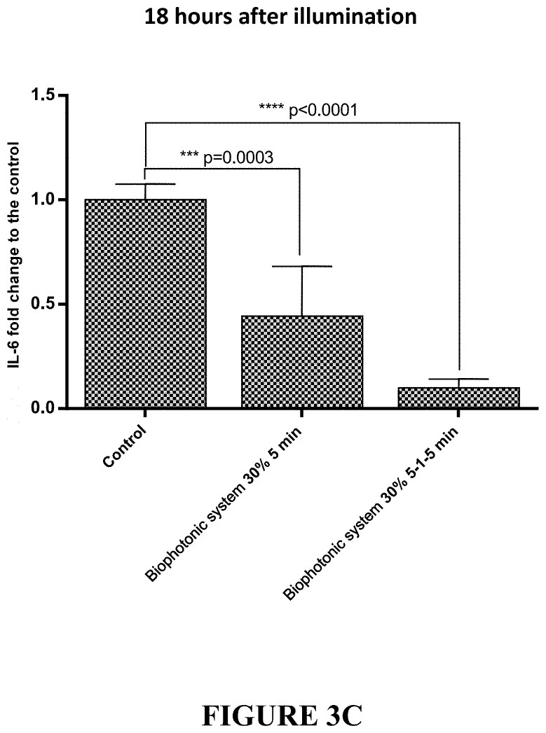

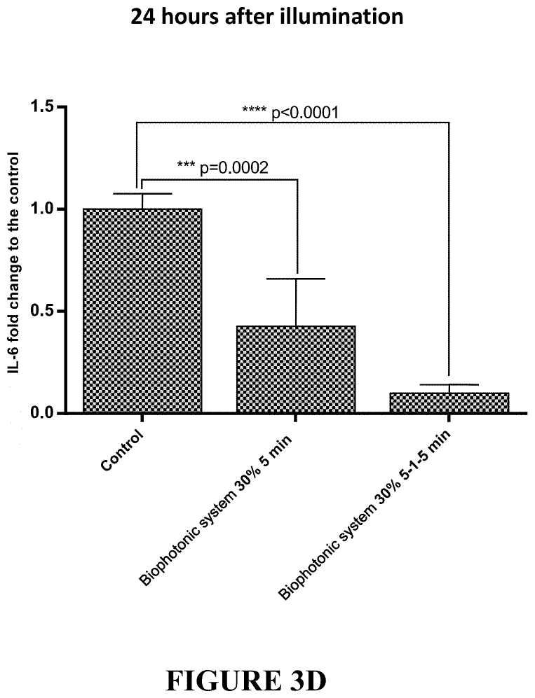

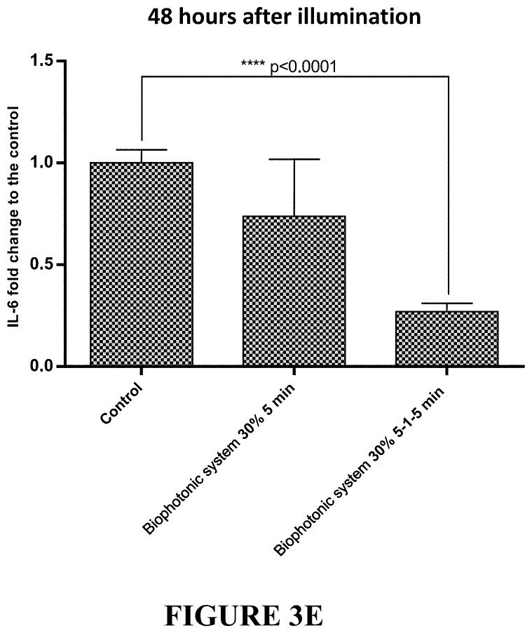

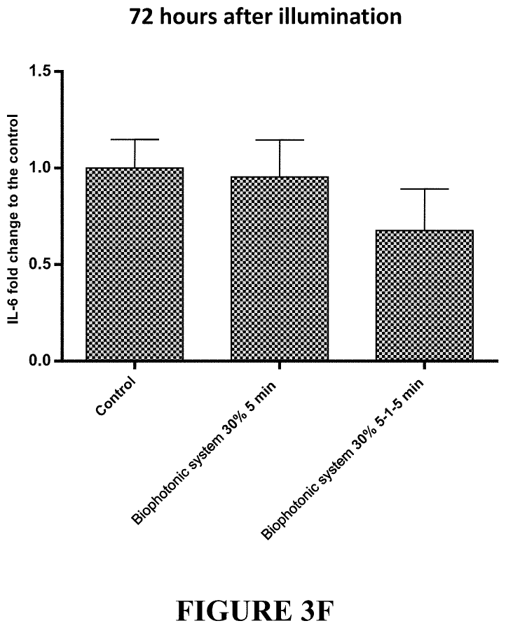

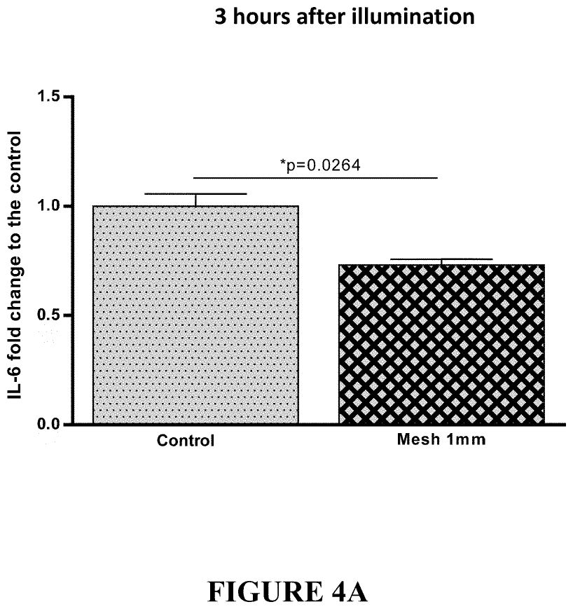

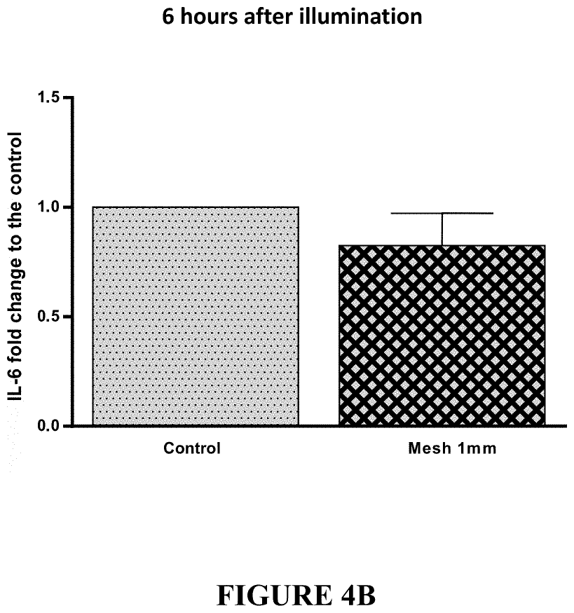

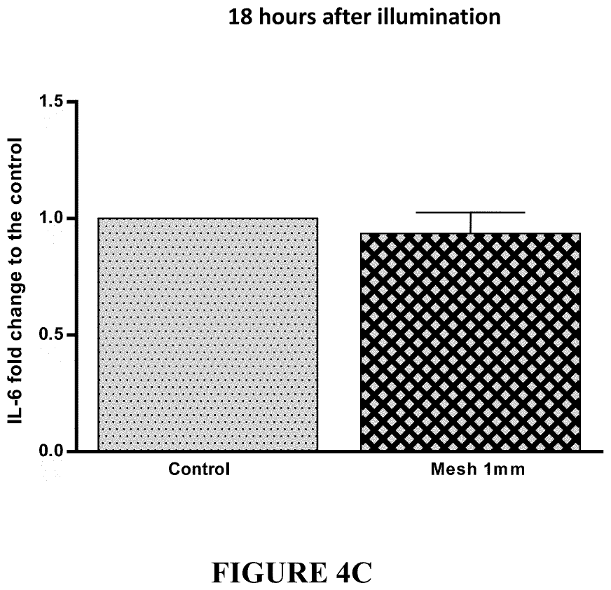

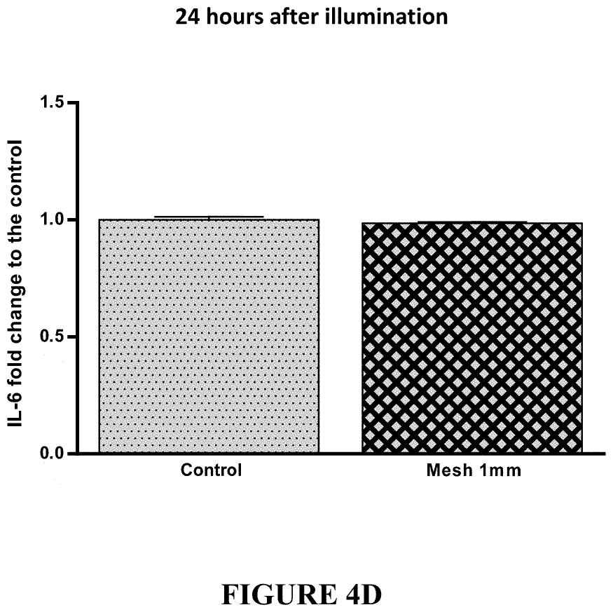

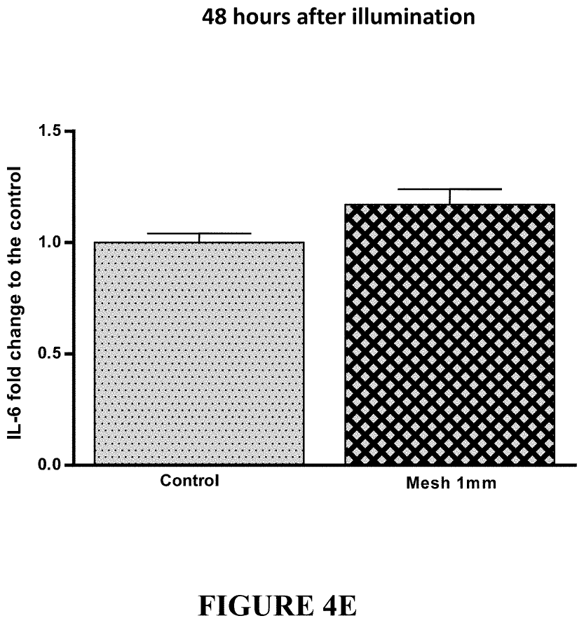

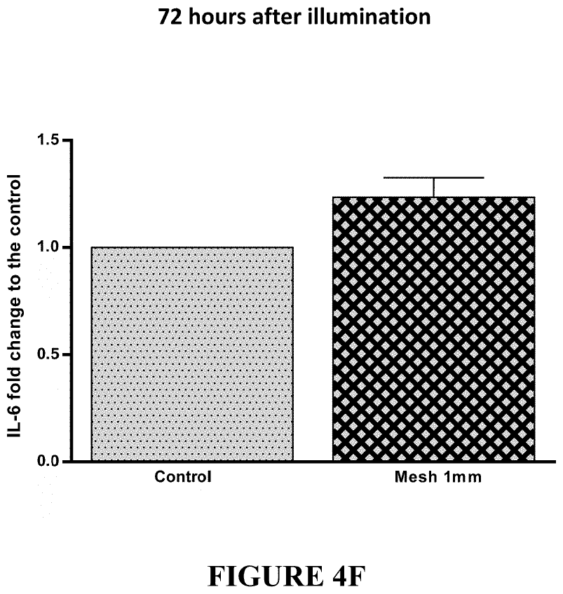

ELISA was performed according to the manufactures' protocol. Samples were diluted in reagent diluent (if needed) for analysis. Absorbance at 450 nm was determined using the Synergy HT microplate reader (Biotek, Winooski, Vt., USA) and corrected for absorbance at 570 nm. Results were analyzed using Excel (Microsoft office 2016). The viability of cultured cells was determined at the end of each experiment by assaying the reduction of (2,3-Bis-(2-Methoxy-4-Nitro-5-Sulfophenyl)-2H-Tetrazolium-5-Carboxanilide) (XTT) to formazan. After treatment, the cells were incubated with XTT solution (final concentration 250 μg/ml) for 2 hours at 37° C. The reduction of live cells was calculated as a percentage of control absorbance in the presence of the medium. Absorbance at 450 nm was determined using the Synergy HT microplate reader (Biotek, Winooski, Vt., USA). Results were analyzed using Excel (Microsoft office 2016). Statistical analyses were performed using 2-way ANOVA or one-way ANOVA with Tukeys multiple comparisons test for DRC and TC and cell proliferation assays respectively. To determine if the hydrogel mesh 50/50 has any anti-inflammatory effects when applied in combination with KT-L™ LED Lamp on IL-6 induction, the healthy DHF cells were stimulated with 20 ng/ml of IL-1α/β as described above. The effect of the formulation was observed in a time-dependent manner. The results are presented in FIGS. 3A-3F as well as in FIGS. 4A-4F. There was a statistically significant decrease of IL-6 from 3 to 24 hours of exposure when cells were treated for 5 minutes with the biophotonic mesh+hydrogel 50/50 compared to the control cells that did not receive any treatment. At 48 hours and at 72 hours, IL-6 release was back to the baseline. These results suggest a good modulation of the inflammation by the biophotonic mesh+hydrogel in 50/50 within the first 24 hours of treatment. After a pre-stimulation overnight with 20 ng/ml of IL-1α/β, DHF cells were treated with the absorbent biophotonic system comprising the mesh+hydrogel in 50/50 combination with KT-L™ Lamp. After illumination, cells were again incubated with IL-1α/β cocktail for 3, 6, 18, 24, 48, or 72 hours. Data show a statistically significant IL-6 reduction in the 5-minute treatment with the biophotonic mesh+hydrogel in 50/50 combination during the first 24 h compared to the control. The production of the pro-inflammatory mediator (IL-6) was assessed by ELISA. The result represents the mean of three independent experiments performed in quadruplicate ±SD. These results show that the absorbent biophotonic system of the present technology promotes healing and/or the treatment of a wound.

### Example 7

### In vivo Testing of an Absorbent Biophotonic Fiber System

Wounds (3) were created (5 cm in diameter) on a swine, and with a depth of approximately 3 mm. A biopsy was taken from each wound when the wounds were created. The biopsy was cut into pieces: i) two pieces from the cut biopsy were processed in Trizol and stored in liquid nitrogen; and ii) one piece from the cut biopsy was processed in Formalin and used further analysis. The day the wounds were created, a biopsy of a healthy skin (control) was taken as well as a biopsy from the wounded area. Before treatment with hydrogel, a litmus paper was applied on the open area to determine the pH of the wound. The same application was repeated at each dressing changed. The following treatment systems were tested applied to cover the peripheral wound and the created wound:

Treatment #1: Standard of Care (SOC): Normal Gauze was applied and ensured that the gauze stayed on the swine between each treatment. No illumination was applied.

Treatment #2: absorbent biophotonic woven fiber system as defined in Example 1 was applied and a 5 min illumination with KT-P50™ lamp (KLOX Technologies Inc., Laval, Calif.) was applied at a distance of about 1.5-3.0 cm from the wound.

Treatment #3: absorbent biophotonic non-woven fiber system as defined in Example 6 was applied and a 5 min illumination with KT-P50™ lamp (KLOX Technologies Inc., Laval, Calif.) was applied at a distance of about 1.5-3.0 cm from the wound.

During the illumination period, the treated wound received illumination, while the control wounds were covered with multiple layers of drapes used during operations. The control wounds were covered so that the wounds were not exposed to blue light. Pictures of the wounds were taken with a digital imaging camera when the wound was created and before each treatment, as well as measurements made using the software to define: Area, Area reduction, Perimeter, Length, Width, Max Depth, Mean Depth and Volume. Each wound was scored using the Draize score and Modified Hollander Cosmesis Score in order to characterize the healing parameters. After the illumination was completed, the absorbent biophotonic fiber system was left in place on the wound and covered with Millipore tape and bandage. The treatment was repeated twice weekly (repeated steps 2-10) and this was continued until all the wounds had reached full closing as determined by the Scoring System. When a wound was fully closed, a biopsy sample was collected from the wound area. The process and storage of the biopsy was done as listed in Step 2. Table 9 below shows progression of wound healing after 1, 5, 8, 12, 15, 19, 26, 29 and 33 days of treatment with the standard of care; Table 10 shows progression of wound healing after 1, 5, 8, 12, 15, 19, 26, 29 and 33 days of treatment with Treatment #2; and Table 11 shows progression of wound healing after 1, 5, 8, 12, 15, 19, 26, 29 and 33 days of treatment with Treatment #3. Tables 12 and 13 present the quantity of exudate absorbed by the fiber systems.

TABLE 9
Wound healing progression after the indicated days of standard of care treatment

| | Day 1 | Day 5 | Day 8 | Day 12 | Day 15 | Day 19 | Day 26 |
|---|---|---|---|---|---|---|---|
| Area (cm²) | 19.8 | 21.5 | 15.77 | 6.6 | 4.7 | 2.2 | 1.7 |
| Perimeter (mm) | 163 | 17 | 144 | 96 | 81 | 114 | 55 |
| Max. Depth (mm) | 8 | 6 | 2 | 2 | 2 | 0 | 2 |
| Mean Depth (mm) | 5 | 2 | 0 | 0 | 0 | 0 | 0 |
| Volume (cm³) | 9.5 | 5.1 | 0.3 | 0 | 0.1 | 0 | 0 |
| pH | 8 | 8 | 8 | 7.5 | 8 | N/A | N/A |

| | Day 29 | Day 33 |
|---|---|---|
| Area (cm²) | 1.3 | 0 |
| Perimeter (mm) | 66 | 0 |
| Max. Depth (mm) | 0 | 0 |
| Mean Depth (mm) | 0 | 0 |
| Volume (cm³) | 0 | 0 |
| pH | N/A | N/A |

TABLE 10
Wound healing progression after the indicated days of Treatment #2

| | Day 1 | Day 5 | Day 8 | Day 12 | Day 15 | Day 19 | Day 26 |
|---|---|---|---|---|---|---|---|
| Area (cm²) | 18.2 | 19.8 | 15.8 | 5.9 | 2.7 | 0 | 0 |
| Perimeter (mm) | 156 | 174 | 146 | 90 | 76 | 0 | 0 |
| Max. Depth (mm) | 7 | 4 | 1 | 2 | 1 | 0 | 0 |
| Mean Depth (mm) | 4 | 1 | 0 | 0 | 0 | 0 | 0 |
| Volume (cm³) | 7.7 | 2.8 | 0.1 | 0 | 0.1 | 0 | 0 |
| pH | 8 | 7.5 | 7.5 | 7 | N/A | N/A | N/A |

| | Day 29 | Day 33 |
|---|---|---|
| Area (cm²) | 0 | 0 |
| Perimeter (mm) | 0 | 0 |
| Max. Depth (mm) | 0 | 0 |
| Mean Depth (mm) | 0 | 0 |
| Volume (cm³) | 0 | 0 |
| pH | N/A | N/A |

TABLE 11
Wound healing progression after the indicated days of Treatment #3

| | Day 1 | Day 5 | Day 8 | Day 12 | Day 15 | Day 19 | Day 26 |
|---|---|---|---|---|---|---|---|
| Area (cm²) | 19.4 | 19.4 | 14.2 | 7.6 | 0.9 | 0 | 0 |
| Perimeter (mm) | 164 | 166 | 153 | 103 | 42 | 0 | 0 |
| Max. Depth (mm) | 9 | 8 | 1 | 2 | 0 | 0 | 0 |
| Mean Depth (mm) | 5 | 4 | 0 | 0 | 0 | 0 | 0 |
| Volume (cm³) | 9.2 | 8.1 | 0.3 | 0.1 | 0 | 0 | 0 |
| pH | 8 | 7 | 7 | 7 | N/A | N/A | N/A |

| | Day 29 | Day 33 |
|---|---|---|
| Area (cm²) | 0 | 0 |
| Perimeter (mm) | 0 | 0 |
| Max. Depth (mm) | 0 | 0 |
| Mean Depth (mm) | 0 | 0 |
| Volume (cm³) | 0 | 0 |
| pH | N/A | N/A |

TABLE 12
Quantity of exudate absorbed by the fiber system
after the indicated days of Treatment #2

| | Amount (g) | Amount (g) | Amount (g) |
|---|---|---|---|
| | at Day 12 | at Day 15 | at Day 19 |
| Pre-treatment | 12.7 | 15.8 | 15.7 |
| Post-treatment | 21.8 | 22.6 | 17.6 |
| Difference | 9.1 | 6.8 | 1.9 |

TABLE 13
Quantity of exudate absorbed by the fiber system
after the indicated days of Treatment #3

| | Amount (g) | Amount (g) | Amount (g) |
|---|---|---|---|
| | at Day 12 | at Day 15 | at Day 19 |
| Pre-treatment | 15.8 | 12.8 | 12.8 |
| Post-treatment | 26.8 | 24.2 | 14.2 |
| Difference | 11 | 12 | 1.4 |

Treatment with the absorbent biophotonic fiber systems (Treatments #2 and #3) indicates that at day 5 of the treatment, the wound was half of its initial volume and tissue type was 100% granulation tissue. In addition, the wound closed on day 12 when tissue types were granulation (51%) and epithelial (49%). Rate of closure of this wound was 0.64 cm³/day, whereas the rate of closure of the wound treated with the standard biophotonic formulation (gel) was 0.4 cm³/day (data not shown). In light of these results, treatment with the absorbent biophotonic fiber system accelerates the healing process compared to the standard of care treatment and compared to the standard biophotonic formulations (gel). Wound treated with the absorbent biophotonic system closed about 41% faster than the wounds treated with the standard biophotonic formulations (gel).

### INCORPORATION BY REFERENCE

All references cited in this specification, and their references, are incorporated by reference herein in their entirety where appropriate for teachings of additional or alternative details, features, and/or technical background.

### EQUIVALENTS

While the disclosure has been particularly shown and described with reference to particular embodiments, it will be appreciated that variations of the above-disclosed and other features and functions, or alternatives thereof, may be desirably combined into many other different systems or applications. Also, that various presently unforeseen or unanticipated alternatives, modifications, variations or improvements therein may be subsequently made by those skilled in the art which are also intended to be encompassed by the following embodiments.

1. An absorbent biophotonic fiber system comprising:
at least one biophotonic fiber component; and
at least one absorbent component; wherein the at least one biophotonic fiber component is photo-stimulated upon exposure to light to emit fluorescence.

2. The absorbent biophotonic fiber system according to claim 1, wherein the at least one absorbent component is a hydrogel component.

3. The absorbent biophotonic fiber system according to claim 1, wherein the at least one biophotonic fiber component comprises biophotonic fibers.

4. The absorbent biophotonic fiber system according to claim 3, wherein the biophotonic fibers are woven.

5. The absorbent biophotonic fiber system according to claim 3, wherein the biophotonic fibers are non-woven.

6. The absorbent biophotonic fiber system according to claim 3, wherein the biophotonic fibers comprise light-accepting molecules.

7. The absorbent biophotonic fiber system according to claim 6, wherein the light-accepting molecules are Eosin Y.

8. The absorbent biophotonic fiber system according to claim 6, wherein the light-accepting molecules are Eosin Y and Fluorescein.

9. The absorbent biophotonic fiber system according to claim 1, wherein photo-stimulation of the at least one biophotonic fiber component causes the absorbent biophotonic system to emit fluorescence.

10. The absorbent biophotonic fiber system according to claim 9, wherein the fluorescence emitted has a wavelength ranging from between 400 nm and about 700 nm.

11. The absorbent biophotonic fiber system according to claim 1, wherein photo-stimulation of the at least one biophotonic fiber causes the absorbent biophotonic system to emit fluorescence in the yellow, orange and/or red regions.

12. The absorbent biophotonic fiber system according to claim 3, wherein the biophotonic fibers are composed of nylon.

13. The absorbent biophotonic fiber system according to claim 3, wherein interstices are present between fibers of the biophotonic fibers.

14. The absorbent biophotonic fiber system according to claim 2, wherein the at least one absorbent component comprises hydrogel.

15. The absorbent biophotonic fiber system according to claim 14, wherein the hydrogel comprises a plurality of apertures.

16. The absorbent biophotonic fiber system according to claim 1, having a thickness of between about 0.1 mm and about 10 mm, or between about 1 mm and about 9 mm, or between about 1 mm and about 6 mm, or between about 2 mm and about 6 mm.

17. The absorbent biophotonic fiber system according to claim 1, wherein the at least one biophotonic component has a thickness of between about 0.1 mm and about 3 mm, or between 1 mm and about 3 mm, or between about 1 mm and about 2 mm, or less than about 2 mm, or less than about 1 mm.

18. The absorbent biophotonic fiber system according to claim 1, wherein the at least one absorbent component has a thickness of between about 0.1 mm and about 3 mm, or between 1 mm and about 3 mm, or between about 1 mm and about 2 mm, or less than about 2 mm, or less than about 1 mm.

19. The absorbent biophotonic fiber system according to claim 1, wherein the at least one biophotonic component is disposed between a first absorbent component and a second absorbent component.

20. The absorbent biophotonic fiber system according to claim 1, wherein the at least one biophotonic fiber component is a mesh.

21-36. (canceled)
